(19) United States
(12) Patent Application Publication
(10) Pub. No.: US 2018/0140789 A1
(43) Pub. Date: May 24, 2018

(54) INHALER DEVICE

(71) Applicant: CIPLA EUROPE NV, ANTWERPEN (BE)

(72) Inventors: Frank PIETERS, Antwerp (BE); Geena MALHOTRA, Mumbai (IN)

(21) Appl. No.: 15/572,874

(22) PCT Filed: May 11, 2016
(86) PCT No.: PCT/GB2016/051352

§ 371 Date: Nov. 9, 2017

(30) Foreign Application Priority Data
May 12, 2015 (IN) 1870/MUM/2015

(51) Int. Cl. A61M 15/00

(52) U.S. Cl. CPC A61M 15/0035; A61M 11/06; A61M 15/0021; A61M 15/004

(57) ABSTRACT

An inhaler for delivering a powdered medicament from a container. The inhaler includes a flow channel, an opening means and a housing. The flow channel includes a conduit extending from an inlet to an outlet and includes a medicament introduction region within which a powdered medicament can be introduced. The housing includes a loading member which includes a holder for receiving a medicament container. The loading member can be manually moved between a loading position, in which a container can be manually loaded into, or removed from, the holder, and an inhalation position in which the powdered medicament can be introduced into the flow channel. The movement of the loading member from the loading position to the inhalation position causes the opening means to break a seal on the container, thereby exposing the powdered medicament for introduction into the flow channel.

### FIELD OF INVENTION

The present invention relates to an inhaler device for delivering a powdered medicament to a user from a sealed container. The invention relates in particular to a reloadable, single unit or single dose, inhaler device which receives a sealed container containing the powdered medicament to be delivered to a patient.

### BACKGROUND OF THE INVENTION

Various types of dry powder inhaler devices are known, including single dose, multiple unit dose and multi-dose devices. Single dose inhalers are generally openable such that a capsule, such as a gelatine capsule, containing the medicament can be placed into a receptacle area of the inhaler. The capsule has to be opened or perforated in order to allow part of the inspiratory air stream to enter the capsule for medicament entrainment or to discharge the powder from the capsule during inhalation. Generally this is achieved by opening or perforating the capsule with pins or cutting blades, to enable powder to leave the capsule. After inhalation, the emptied capsule is removed from the inhaler, such that a further capsule can be loaded for subsequent inhalation. Therefore, it is necessary to open the inhaler for insertion and retrieval of the medicament capsules, which is an operation which can be difficult for some patients.

In order for such a dry powder inhaler device to be successful, it is important that as much medicament as possible reaches the lungs of the user and that use of the inhaler is convenient and easy, particularly for users with limited manual dexterity who may find manipulating small gelatine capsules difficult. If the inhaler is not easy to use patient compliance with a prescribed dosage regimen may be reduced which can result in more frequent visits to healthcare workers or healthcare professionals.

Typically, when a powdered substance is inhaled through a mouthpiece or nozzle of a dry power inhaler, a substantial part of the active pharmaceutical agent held in the capsule fails to reach the lungs of the user. In particular, a significant quantity may be deposited in the oral cavity of the user. This can lead to a significant loss of active pharmaceutical agent during inhalation. Problems also exist with the airborne delivery of particulate medicaments in that the particles can agglomerate during storage. Agglomeration of the medicament leads to inefficiency in delivery, as a greater proportion of the medicament fails to reach the lungs due to increased particle mass. Agglomerated particles may also lead to difficulties in medicament absorption through the mucous membranes of the respiratory system and possible irritation.

WO2007/007110 describes an inhaler which addresses the efficient delivery of medication by providing an inhaler which uses airflow directing means to modify the airflow through the inhaler to help enhance medicament delivery to the lungs of a user. To use the inhaler a medicament container must be orientated and arranged between support arms and then pushed towards the inhaler body until the container is pierced. It has been found that such orientation and manipulation can be problematic for some users and requires tight control of manufacturing tolerances.

WO2005/120615 discloses an inhaler with a movable slide which is opened to receive a container of medicament and then pushed closed as the user inhales. As the slide is closed the container is opened by the inhaler and the medicament delivered. However, this requires coordination of the closing of the slide and inhalation which may be problematic for some users.

WO2007144614 discloses a single dose inhaler formed in two parts which are hinged together using an integrally formed living hinge. When closed the two parts define an airflow path including a circulating airflow chamber.

### OBJECT OF THE INVENTION

The invention provides an inhaler for delivering a powdered medicament to a user from a container.

### SUMMARY OF THE INVENTION

An inhaler for delivering a powdered medicament to a user from a container, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

Use of a powdered medicament comprising insulin delivered by an inhaler for the treatment of patients with diabetes mellitus for the control of hyperglycemia, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

A method for treating a patient with diabetes mellitus, the method comprising the steps of administering to a patient in need thereof a powdered medicament comprising insulin from an inhaler, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

Use of the powdered medicament comprising insulin delivered by an inhaler for the treatment of patients with diabetes mellitus for the control of hyperglycemia, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

### DESCRIPTION OF THE INVENTION

The invention provides an inhaler for delivering a powdered medicament to a user from a container, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

The movement of the loading member from the loading position to the inhalation position is configured to cause the opening means to break a seal on a container retained in the holder to thereby expose the powdered medicament for introduction into the flow channel. Providing a loading member which is movable, particularly movable relative to the housing, and which carries a holder, means that the holder can be moved to a position in which a user can more easily arrange a container in the holder, for example in a position away from the body in which the holder may be exposed thereby allowing a user to more easily guide a container into the holder, and also to retrieve a used container from the holder. Once the container is arranged in the holder it is retained by the holder so that the container cannot simply fall out of the holder, but must be removed by a user applying a removal force to the container to separate it from the holder. This means that, once the container is arranged in the holder the inhaler can be moved into a position in which the loading member can be more easily manipulated by a user, without the risk that the container will easily fall from the holder. This means that a user can move the inhaler into a comfortable position to move the loading member into the loading position, change the position of the inhaler to load a container into the holder and then move the inhaler into another orientation to move the loading member to the inhalation position. By retaining the container in the holder against, for example, gravity, such user handing can be conducted without a significant risk that the container will fall from the holder and be lost and so the user can arrange the inhaler in the most comfortable position for them to be able to perform the required actions.

The use of a loading member further means that the inhaler need not be opened to expose internal parts to potential damage, or expose the user to parts that could potentially cause harm. The housing remains intact and a loading member is used to transfer a container to the required location for use.

Once the container is arranged in the holder the loading member can be moved to an inhalation position. In the inhalation position a container retained in the holder is arranged at least partly within the housing, which may render it difficult to access manually. The container may be located substantially entirely within the housing. The inhalation position of the holder is determined by the movement of the loading member so a container in the holder can be reliably and conveniently moved from a loading position to a desired inhalation position. The movement of the holder is constrained by the loading member in such a way that it facilitates the breaking of a seal on the container as the container is moved into the inhalation position. The loading member may be coupled to the housing and may be coupled such that the holder follows a predetermined path as it moves to the inhalation position, for example the holder may follow a defined substantially arcuate path, possibly about a loading axis, and this can enhance the consistency of the breaking of the seal by the opening device as the holder follows substantially the same path each time it is moved.

The opening means can be any suitable device, element or combination of elements which is able to break a seal on the container and may comprise, for example, one or more piercing members, such as needles or pins, one or more cutting members, such as blades. The opening device may be movable relative to the housing, but is preferably substantially fixed relative to the housing as this facilitates construction and reduces the number of moving parts that must be aligned to achieve reliable breaking of the seal. The opening device may be a piercing member arranged to pass through and break a seal of a container retained in the holder.

The piercing member could be substantially solid, but may include a piercer conduit therethrough. The piercer conduit may extend from the medicament introduction region of the flow channel to a piercer inlet. The piercer inlet is preferably arranged within a container retained in the holder when the loading member is in the inhalation position. Such an arrangement allows powdered medicament from within said container to be drawn through the piercer conduit and into the flow channel.

The piercing member may include a seal spreader portion which, in the inhalation position is arranged within a container arranged in the holder. The seal spreader portion includes an outer surface region which is enlarged compared with a body region of the piercing member. As the seal spreader portion passes though the seal it causes a hole in the seal created by the piercing element to be enlarged so that the seal spreader portion can pass through. Once the seal spreader portion has passed through the seal a body region of the piercing member may be located in the enlarged hole. The seal spreader portion enlarges the hole such that a flow path into the container past the seal is created between the seal and the piercing member. Such an arrangement allows a single piercing member to be inserted into the container to create both a flow path out of the container, through the piercer conduit, and a flow path into the container past the seal, through the enlarged hole created by the seal spreader portion without the need to remove the piercing member from the hole.

The piercing member may taper outwardly from a piercer inlet to the seal spreader portion and then taper inwardly to a body region of the piercing member.

The flow channel may extend substantially along a channel axis and may be substantially linear. The conduit in the flow channel may also extend substantially along the channel axis and may be substantially linear and may be substantially coaxial with the flow channel. The piercer conduit may extend substantially along a piercer axis, the channel axis being substantially perpendicular to the piercer axis. The piercer conduit may be substantially linear. A substantially linear flow channel and piercer channel, coupled with a perpendicular arrangement can facilitate manufacture. The piercing member may also extend substantially along the piercer axis and may be substantially coaxial with the piercer conduit. The medicament inhalation region may be located between the conduit inlet and the conduit outlet as this means that the medicament is not introduced at, for example, the inlet of the conduit and this increases the options available for arranging elements of the inhaler.

The holder may comprise one or more structures which cooperate with the loading member to retain a container. For example the holder may comprise an aperture through the loading member within which a container can be arranged. The one or more structures may be shaped to fit with, or around, the shape of the container to be retained. For example, for a container with a substantially circular cross section, a substantially circular aperture might be used or the one or more structures could define all or part of a substantially cylindrical wall.

The holder may comprise at least two fingers extending from the loading member. The fingers may be configured to engage an outer surface of a container arranged in the holder to help retain the container in the holder. One, or all, of the fingers, and/or the loading member from which they extend, may be resiliently deformable and may have a rest state which would not allow a container to be accepted into the holder. This means that the fingers or loading member must be deformed to allow a container to be received in the holder and this can enhance the retaining forces applied on the container by the fingers. In some embodiments a container can be retained in the holder by friction forces alone, although clips, catches, detents or the like could also be used. Alternatively, or additionally, the container may be resiliently deformable to serve the same purpose.

In some embodiments the holder is formed by two fingers. Each finger may define part of a substantially incomplete substantially cylindrical wall within which a container can be received.

If the container does not have a constant cross section along its length the holder may be shaped to include parts that substantially correspond to the external shape of a container, for example, if the container narrows towards its closed end, the holder may include lower elements which narrow a portion of the cylinder adjacent the loading member within the incomplete cylindrical wall such that the internal shape of the holder substantially corresponds to an external shape of said container. This can help to prevent a user from inserting a container into the device upside down. The holder can also include orientation elements which cooperate with a container to restrict the orientation of a container within the holder. In some embodiments the container within the holder may be able to be rotated only about its container axis. Having a container which narrows at the closed end can facilitate introduction of the container into the holder.

The loading member may be coupled to the inhaler such that it can rotate about a loading axis between the loading and inhalation positions. The loading axis may be substantially perpendicular to an axis along which the flow channel extends such as, for example the channel axis which is an axis along which the piercer conduit extends or the piercer axis or both. The coupling may be such that the loading member can pivot about the loading axis. The loading member may extend away from the loading axis to the holder thereby allowing the holder to be moved away the housing when the loading member is moved to the loading position. The distance between the loading axis and holder can also be used to provide leverage for moving the loading member and breaking a seal on a container. In the loading position the loading member may extend away from the housing in a direction substantially parallel with the piercer axis and in the inhalation position the loading member may extend substantially parallel with the conduit axis.

The housing may substantially enclose the flow channel and may comprise a mouthpiece opening at or adjacent the outlet, an intake opening at or adjacent the inlet and a container opening for accepting at least a portion of container retained in the holder when the loading member is moved to the inhalation position. The housing adjacent the mouthpiece opening may be adapted for use as a mouthpiece by a user, but could also be adapted for nasal use if desired. The container opening may open into a recess within the housing within which a portion or, or substantially all of, a container retained in the holder can be located when the loading member is in the inhalation position. A mouthpiece may include indicia, which may be printed, etched or moulded, to indicate to a user/patient or caregiver the extent, or distance, to which they should place the mouthpiece into their mouth to enable smooth inhalation of the medicament.

The loading member may substantially cover the container opening when in the inhalation position. Although the loading member may cover the container opening, it may not seal the container opening. By not sealing the container opening airflow through the container opening can be used to provide airflow into the container. Covering the container opening helps to prevent foreign bodies from entering the inhaler and can also help to prevent damage to an exposed opening device.

The housing may be integrally formed with the flow channel, or may comprise one or more sections. In some embodiments the housing comprises a front housing which includes the mouthpiece opening, and a rear housing which includes the intake opening and container opening. A two part housing that contains a flow channel can facilitate manufacturing of the inhaler by enabling the flow channel to be easily located within the housing. The loading member may be pivotally coupled to the rear housing as this provides a convenient location for coupling the loading member and helps to define the front and rear of the device.

The inclusion of a housing enables the external shape of the inhaler to be controlled, not simply for aesthetic reasons, but the use of a housing, particularly one which is not formed integrally with the flow channel, allows changes to be made without the need to alter the flow channel. A housing can also be used to provide external features such as grip enhancing portions, guide indicia, or a comfortable mouthpiece shape, and can also be used to conceal, or protect, potentially fragile parts, or parts which might cause damage or catch in things. For example, the inclusion of a container opening in the housing allows the container to be received in that opening. Since the container can enter the housing, this allows the opening means to be arranged within the housing. For example, a piercing member need not protrude out of the container opening meaning that it is less likely to be damaged or to catch in pockets or clothing, but the piercing member can still break the seal on a container when the container is moved into the container opening. The housing could include indicia, which may be printed, etched or moulded, to indicate how to hold the device during inhalation. For example the housing may include indicia which indicate in which orientation the device should be held during inhalation. Some indicia may be visible to a user during inhalation only if the device is used correctly, or only if the device is used incorrectly. Indicia may be included that indicates how to operate and load the inhaler device. The housing may also be coloured to aid identification, for example if an inhaler device is particularly intended for use with a particular medicament.

The flow channel and front and rear housings may be secured together using snap fit connections. The loading member may be coupled to the housing, particularly a rear housing using a snap fit connection.

The conduit in the flow channel may comprise a flow restriction in, or adjacent, the medicament introduction portion such that the flow channel includes a region of reduced pressure in the medicament introduction portion when air flows through the flow channel from the inlet to the outlet. The reduced pressure in the medicament introduction portion causes air to be drawn to the medicament introduction portion, for example through a piercer conduit. The restriction may be, for example, the restriction in the form of a venturi.

The inhaler may include flow directing means for directing air flowing into the flow channel in a pre-selected direction. The flow directing means may restrict the amount of air entering, or leaving, the flow channel. Preferably the airflow directing means comprises at least one turbulence member to impart turbulence and/or directional flow to air flowing into or out of the first air passageway. The, or each, turbulence member may direct substantially all the air flow in substantially the same direction, for example by adding a circumferential flow element to the flow within the flow channel to impart a helical or whirling motion to air entering the flow channel. The, or each, turbulence member may direct the airflow in opposing directions such that there is some cancelling out of the imparted flow direction, which can further enhance turbulence.

The flow directing means may comprise an insert arranged at or adjacent to the inlet end of the flow channel. The insert may include a plurality of flow directing channels therethrough and these channels may be defined by turbulence members. Air passing through the flow channel must pass through the flow directing channels causing the air to flow in a particular direction. Preferably the flow channels impart a swirling or rotational flow to the air entering the flow channel. Preferably the swirling or rotational flow moves along the flow channel and rotates around the channel axis.

The inhaler described above is preferably a reloadable single unit dry powder inhaler. A reloadable single unit dry powder inhaler is able to be loaded with a container which includes a single dose, or part of a single dose, of the powdered medicament, deliver that dose and allow the used container to be replaced with a new container as required.

The invention also provides an inhaler kit comprising an inhaler and a container, the inhaler being as described above and the container comprising a body and a seal, wherein the body includes a cavity containing a powdered medicament and the seal closes the cavity.

The body of the container may have a substantially cup shaped form and may extend along a container axis from an open end having a rim to a closed end. The exterior of the container body may be substantially circular in cross section perpendicular to the container axis. The container may narrow as it extends towards the closed end. The interior of the container body defines a cavity which is closed by the seal. The seal may be attached to the rim. The container may be substantially rotationally symmetrical about the container axis.

The container body may be formed from a material including a plastic material, such as a plastic laminate, which may include a metallic layer. The seal may be formed from a material including a metal, for example a foil, which may be laminated with one or more plastic materials.

The container body and/or the seal may be provided in any suitable colour, or combination of colours. Such colours could be used to convey information to a user, for example particular medicaments may be allocated a pre-determined colour so that a patient is sure that they are taking the correct medication. The container and/or seal may further include printed, etched or moulded indicia which provide information to a user. The container may be colour coordinated with the housing of an associated inhaler device to assist a user in selecting the appropriate inhaler for a particular medicament.

It is preferred that the exterior surface of the container and the shape of the holder cooperate to retain the container in the holder. These shapes may also cooperate to prevent the container from being received in the holder in an incorrect orientation as discussed above.

It should be noted that inhaler of the present invention as described above is particularly suitable for use with the containers described above.

The invention also provides a method for using an inhaler kit, the inhaler kit being as described above, and the method including the steps of:
manually moving the loading member into a loading position;
loading the container into the holder and using the holder to retain the container;
moving the loading member into an inhalation position in which a powdered medicament which is stored within the container can be introduced into the flow channel;
causing the opening means to break the seal on the container as the loading member is moved from the loading position to the inhalation position;
causing air to flow through the flow channel from the inlet to the outlet;
allowing the powdered medicament from the container to enter the flow channel and become entrained in the air flow as a result of the air flow through the flow channel;
manually moving the loading member from the inhalation position to the loading position while retaining the container in the holder; and
manually removing the container from the holder.

The invention also provides a method for loading a container into an inhaler, the inhaler being as described above and comprising a flow channel, an opening means and a housing, the flow channel comprising a conduit extending from an inlet to an outlet and including a medicament introduction region within which a powdered medicament can be introduced into the flow channel, the housing including a loading member coupled thereto, the loading member including a holder for retaining a container containing the powdered medicament and the method comprising the steps of:
providing a container of a powdered medicament, the container comprising a body and a seal, wherein the body includes a cavity containing a powdered medicament and the seal closes the cavity;
manually moving the loading member into a loading position;
loading the container into the holder and using the holder to retain the container;
moving the loading member into an inhalation position in which a powdered medicament stored within the container can be introduced into the flow channel; and
causing the opening means to break the seal on the container as the loading member is moved from the loading position to the inhalation position.

The invention also provides a method for removing an opened container from an inhaler following use, the inhaler being as described above and comprising a flow channel, an opening means and a housing, the flow channel comprising a conduit extending from an inlet to an outlet and including a medicament introduction region within which a powdered medicament can be introduced into the flow channel, the housing including a loading member coupled thereto, the loading member including a holder for receiving and retaining a container, the inhaler having an opened container retained in the holder, the container comprising a body and a broken seal, wherein the body includes a cavity and the seal closed the cavity before it was broken and the method comprising the steps of:
manually moving the loading member from the inhalation position to the loading position while retaining the container in the holder; and
manually removing the container from the holder.

By providing a loading member which can be moved between a loading position and an inhalation position the use of an inhaler can be significantly facilitated. The loading member can be moved into the loading position in which convenient access to the holder is possible. A container can then be easily loaded into the holder and a seal of the container broken as the loading member is moved to an inhalation position. The inhalation position may place the container in a hard to access location which would present difficulties for some users if they were to try to manually insert or remove the container. The use of the loading member and holder means that the container can be more consistently presented to the opening means and so a more reliable and consistent breaking of the seal may be possible. This can be particularly important if the airflow is going to pass through the break in the seal as differences in the quality of the seal break could have significant effects on the air flow into, or out of, the container.

It should be noted that the term medicament as used herein refers not only to a formulation including a therapeutically active agent, but also to a placebo of such a formulation. The powdered medicament may be provided in the form of a formulation which comprises an active pharmaceutical agent alone, or may include drug particles and carrier particles, for example micronized crystalline drug and a lactose carrier.

It should be noted that the term active pharmaceutical agents as used herein refers to one or more agents, comprising, but not limited to, hormones, corticosteroids, anticholinergic agents, β2 agonists, anticoagulants, immunomodulating agents, vaccines, cytotoxic agents, antibiotics, vasoactive agents, neuroactive agents, anesthetics or sedatives, steroids, decongestants, antivirals, antisense, antigens and antibodies.

Particularly, these active pharmaceutical agents include insulin, such as recombinant human insulin, lispro, aspart, glulisine, detemir insulin, NPH insulin (Neutral Protamine Hagedorn), insulin degludec, glargine insulin heparin (including low molecular weight heparin), formoterol, AR-formoterol, fenoterol, pirbuterol, carmoterol, indacaterol, vilanterol, abediterol, salbutamol, levosalbutamol, olodaterol, salmeterol, tiotropium, ipratropium, oxotropium, glycopyrronium, umeclidinium, aclidinium, budesonide, R(+) budesonide, mometasone, ciclesonide, fluticasone propionate, fluticasone furoate, fluticasone valerate, beclomethasone, betamethasone, calcitonin, felbamate, terbutaline, sumatriptan, parathyroid hormone and active fragments thereof, parathyroid hormone related protein, growth hormone, erythropoietin, AZT, DDI, granulocyte macrophage colony stimulating factor (GMCSF), lamotrigine, chorionic gonadotropin releasing factor, luteinizing releasing hormone, beta-galactosidase, exendin, vasoactive intestinal peptide, and argatroban. Antibodies and fragments thereof can include, in a non-limiting manner, antiSSX-241-49 (synovial sarcoma, X breakpoint 2), anti-NYES0-1 (esophageal tumor associated antigen), anti-PRAME (preferentially expressed antigen of melanoma), anti-PSMA (prostate-specific membrane antigen), anti-Melan-A (melanoma tumor associated antigen), anti-tyrosinase (melanoma tumor associated antigen) follicle stimulating hormone (FSH), glucagon-like peptide-(GLP-1), oxyntomodulin, peptide YY, interleukin, 2-inducible tyrosine kinase, Bruton's tyrosine kinase (BTK), inositol-requiring kinase 1 (IRE!), or analogs, active fragments, PC-DAC-modified derivatives, or 0-glycosylated forms thereof.

It should be noted that the term carrier particles as used herein refers to one or more carbohydrates, e.g., monosaccharides such as fructose, galactose, glucose, D-mannose, sorbose, xylulose and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; amino acids, such as glycine, arginine, aspartic acid, glutamic acid, cysteine, lysine, and the like; organic salts prepared from organic acids and bases, such as sodium citrate, sodium ascorbate, magnesium gluconate, sodium gluconate, tromethamine hydrochloride, and the like; peptides and proteins, such as aspartame, human serum albumin, gelatin, and the like; and alditols, such as sorbitol, mannitol, xylitol, and the like.

The powdered medicament within a container, which is to be delivered by the inhaler, may comprise insulin, particularly human insulin. The powdered medicament may be manufactured by a process which comprises the steps of dissolving insulin, preferably human insulin, in a suitable solvent, adding a carrier and then spray drying the resulting solution to form the powdered medicament.

The inhaler may be used to deliver to a patient a powdered medicament from a container, the powdered medicament comprising insulin, particularly human insulin, for the treatment of patients with diabetes mellitus, particularly for the control of hyperglycemia.

The present invention also provides a powdered medicament comprising insulin delivered by an inhaler for the treatment of patients with diabetes mellitus for the control of hyperglycemia, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

The present invention also provides a method for treating a patient with diabetes mellitus, the method comprising the steps of administering to a patient in need thereof a powdered medicament comprising insulin from an inhaler, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

The present invention also provides the use of the powdered medicament comprising insulin delivered by an inhaler for the treatment of patients with diabetes mellitus for the control of hyperglycemia, the inhaler comprising a flow channel, an opening means and a housing, the flow channel comprising a conduit extending from an inlet to an outlet and including a medicament introduction region within which a medicament can be introduced into the flow channel, the inhaler including a loading member coupled to a portion thereof, the loading member including a holder for receiving and retaining a medicament container, the loading member being manually movable by a user between a loading position, in which the loading member extends away from the housing and the holder is arranged outside of the housing such that a container can be manually loaded into, or removed from, the holder by a user, and an inhalation position in which a container retained in the holder is at least partially within the housing and a powdered medicament stored in a container retained in the holder can be introduced into the flow channel, wherein the movement of the loading member from the loading position to the inhalation position causes the opening means to break a seal on the container, thereby exposing the powdered medicament for introduction into the flow channel.

It should be understood that throughout this specification and in the claims that follow, unless the context requires otherwise, the word “comprise”, or variations such as “comprises” or “comprising”, implies the inclusion of the stated integer or step, or group of integers or steps.

The invention will now be described by way of example only with reference to the following figures in which:

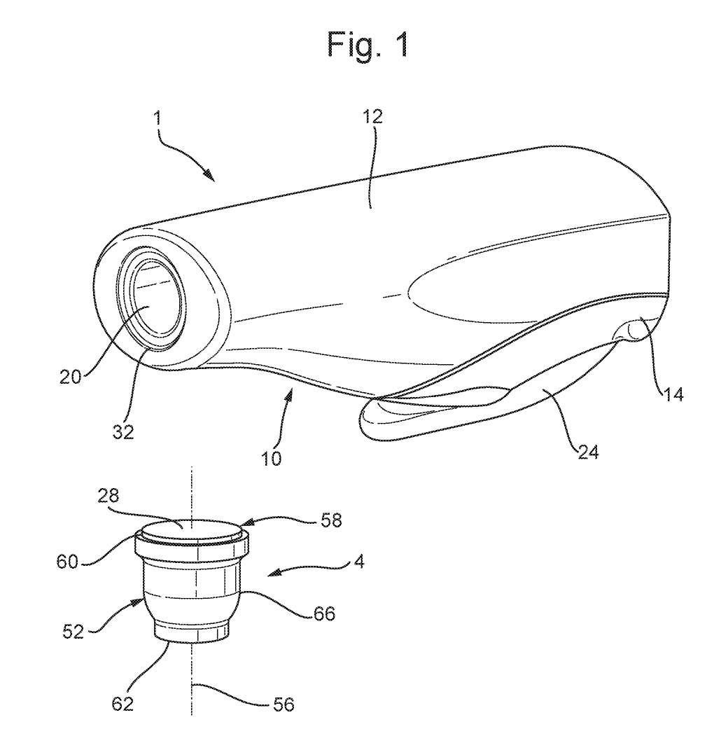

FIG. 1 shows a perspective view of an inhaler and a sealed container;

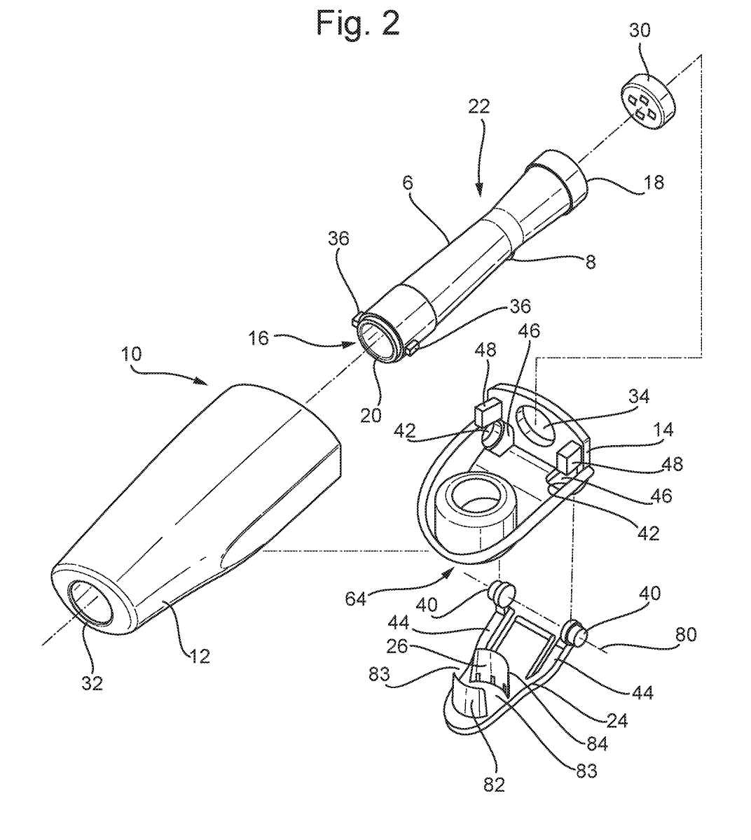

FIG. 2 shows an exploded view of the components of the inhaler of FIG. 1;

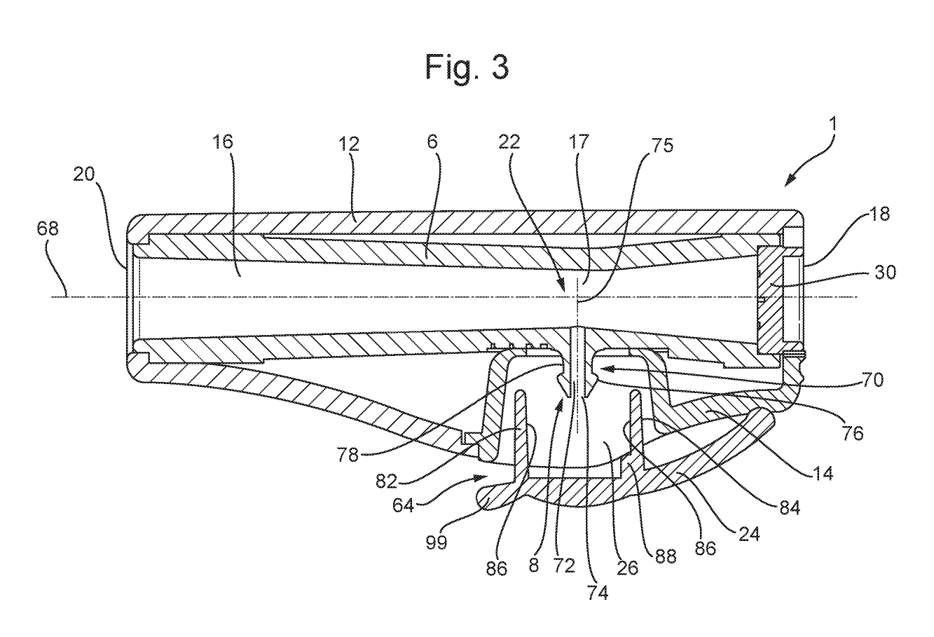

FIG. 3 shows a cross section through the inhaler of FIG. 1;

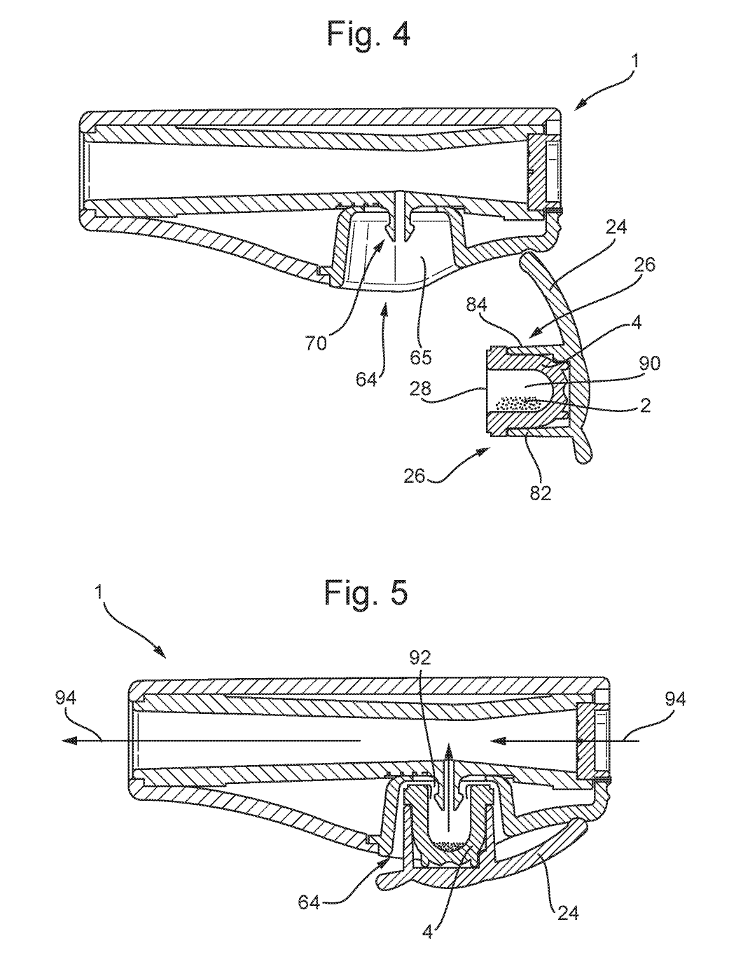

FIG. 4 shows a cross section through the inhaler and container of FIG. 1 with the loading member in a loading position before use;

FIG. 5 shows a cross section through the inhaler and container of FIG. 1 with the loading member in an inhalation position;

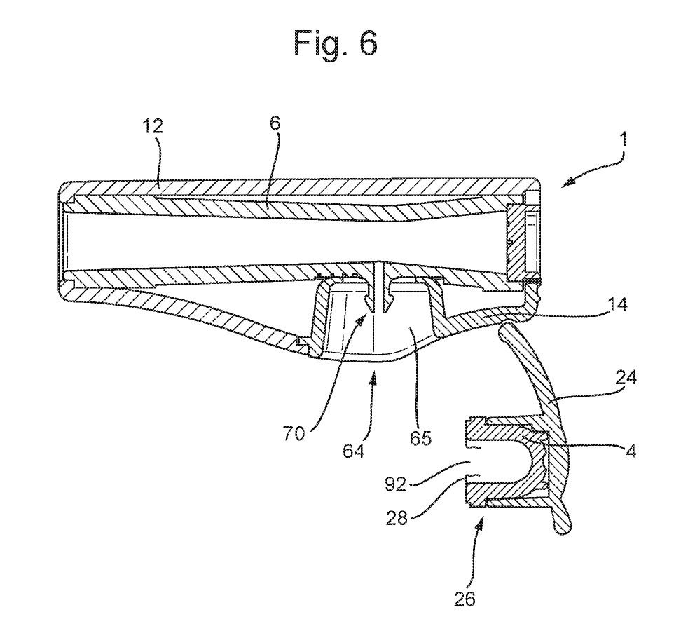

FIG. 6 shows a cross section through the inhaler and container of FIG. 1 with the loading member in a loading position after use;

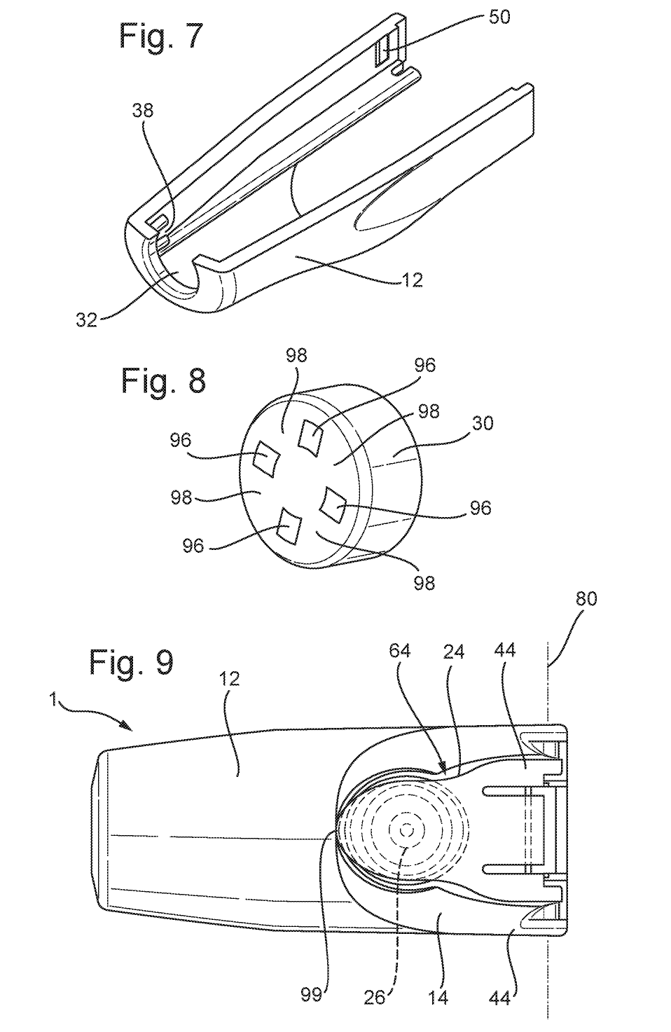

FIG. 7 shows a perspective view of the flow directing means of the inhaler of FIG. 1;

FIG. 8 shows a partial cutaway view of the front housing of the inhaler of FIG. 1;

FIG. 9 shows a view from above the loading member of the inhaler of FIG. 1; and

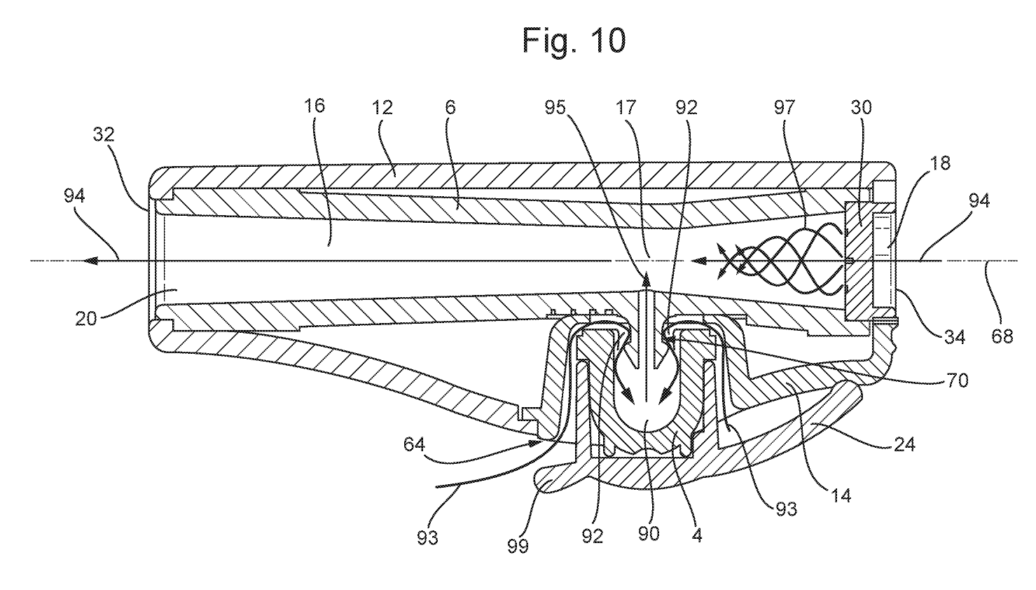

FIG. 10 shows an enlarged view of FIG. 5.

FIG. 1 shows an inhaler 1 for delivering a powdered medicament 2 (not shown in this Figure) to a user from a container 4.

FIG. 2 shows an exploded view of the components of the inhaler 1 of FIG. 1 and provides an indication of how they fit together. The inhaler 1 comprises a flow channel 6, an opening means 8 (shown better in later Figures) and a housing 10. The housing 10 comprises a front housing 12 and a rear housing 14. The flow channel 6 includes a conduit 16 extending from an inlet 18 to an outlet 20 and includes a medicament introduction region 22 (shown better in later Figures) within which a powdered medicament 2 (shown in later Figures) can be introduced into the flow channel 6. The housing 10 includes a loading member 24 coupled thereto. The loading member 24 includes a holder 26 for receiving and retaining a medicament container 4. The loading member 24 is manually movable by a user between a loading position, in which a container 4 can be manually loaded into, or removed from, the holder 26, and an inhalation position in which a medicament 2 stored in a container 4 retained in the holder 26 can be introduced into the flow channel 6. The movement of the loading member 24 from the loading position to the inhalation position causes the opening device 8 to break a seal 28 on the container, thereby exposing the medicament 2 for introduction into the flow channel 6. The inhaler 1 also includes a flow directing means 30 which diverts and directs air flowing into the conduit 16 of flow channel 6 through the inlet 18.

The housing 10 includes a mouthpiece opening 32 in the front housing 12 which communicates with the outlet 20 of the conduit 16 of the flow channel 6 and an intake opening 34 in the rear housing 14 communicating with the inlet 18. The rear housing 14 also includes a container opening 64 into which a container 4 retained in the holder 26 can be inserted.

The components fit together as indicated in FIG. 2, with the flow directing means 30 fitting into the inlet 18 of the conduit 16 and the flow channel 6 fitting into the front housing 12. The rear housing 14 is attached to the front housing 12, substantially closing the housing 10, and the loading member 24 couples to the rear housing 14.

The flow channel 6 is seated and held within the housing 10 by externally directed tabs 36 adjacent the outlet 20 engaging with corresponding slots 38 (see FIG. 7) in the front housing 12, by the outlet 20 of the conduit 16 extending into the mouthpiece opening 32 and by the engagement of the flow directing means 30 in the inlet 18 and intake opening 34.

The loading member 24 couples to the rear housing 14 through the engagement of opposing axles 40 in corresponding recesses 42 in the rear housing 14. The axles 40 are mounted on resiliently deformable legs 44 to allow the axles 40 to pass through axle openings 46 in the rear housing 14 and reach the recesses 42. The axles 40 are arranged along a loading axis 80, and when engaged with the recesses 42 of the rear housing 14 the loading member 24 is able to pivot on the axles 40 about the loading axis 80.

The front housing 12 is secured to the rear housing 14 using snap fit catches 48 which extend from the rear housing 14 and engage with cut-outs 50 (see FIG. 7) in the front housing 12.

The container 4 comprises a body 52 and a seal 28. The body 52 includes a cavity 90 (shown in later Figures) containing a powdered medicament 2. The seal 28 closes the cavity 90. The body 52 of the container 4 is substantially cup shaped and extends along a container axis 56 from an open end 58 having a rim 60, to which the seal 28 is attached, to a closed end 62. An exterior surface 66 of the container body 52 is substantially circular in cross section perpendicular to the container axis 56 and the container 4 narrows as it extends towards the closed end 62. The container 4 is substantially rotationally symmetrical about the container axis 56.

FIG. 3 shows a cross section through the inhaler 1 of FIG. 1. From this figure it can be seen that the conduit 16 in the flow channel 6, and the flow channel 6 extends substantially coaxially with the conduit 16 along a channel axis 68. The opening means 8 is in the form of a piercing member 70 arranged to break the seal 28 of a container 4 in the holder 26 when the loading member 24 is moved to the inhalation position (best shown in later figures).

The piercing member 70 extends outward from the flow channel 6 into a recess 65 within the housing 10 adjacent to the container opening 64 and is directed towards the container opening 64 defined in the housing 10. The piercing member 70 includes a piercer conduit 72 extending from the medicament introduction region 22 of the conduit 16 of the flow channel 6 to a piercer inlet 74. The piercer conduit 72 extends along a piercer axis 75 which extends perpendicular to the channel axis 68.

The piercing member 70 includes a seal spreader portion 76 which comprises an outer surface region which is enlarged compared with a body region 78 between the seal spreader portion 76 and the flow channel 6.

The holder 26 comprises two fingers 82,84 which define a substantially incomplete substantially cylindrical wall within which a container 4 can be arranged. The fingers 82,84 are shaped such that a portion of their internal surface 86 corresponds with a portion of the external surface 66 of a container 4 to increase the area of contact between the two components when the container 4 is arranged in the holder 26 and thereby assist in retaining the container 4 in the holder 26. At least one of the fingers 82,84 includes a projection 88 which narrows the holder 26 so that the container 4 can only fit in the holder one way up (with the seal 28 facing away from the loading member 24).

The flow channel 6 comprises a conduit 16 which extends along the channel axis 68 from the inlet 18 to the outlet 20. The cross sectional area of the conduit 16 decreases from the inlet 18, and from the outlet 20, towards a restricted portion 17 in the medicament introduction region 22 such that the conduit 16 forms a venturi.

FIGS. 4, 5 and 6 show a use sequence of the inhaler 1.

FIG. 4 shows the inhaler 1 with the loading member 24 in a loading position. The loading member 24 extends away from the housing 10. A container 4 with a powdered medicament 2 in its cavity 90 has been arranged in the holder 26 and is retained therein by the fingers 82,84. At least one of the container 4, fingers 82,84 and loading member 24 is resiliently deformed as the container 4 is inserted into the holder 26 to enhance the frictional fit of the container 4 in the holder 26.

From this position the loading member 24 is rotated, or pivoted about the loading axis 80 which extends perpendicular to the channel axis 68 and the piercer axis 70 (so extends into the page as shown) to reach the inhalation position as shown in FIG. 5. The perpendicular arrangement of the channel axis 68, the piercer axis 70 and the loading axis 80 contributes to a compact layout and facilitates consistent piercing.

FIG. 5 shows a cross section through the inhaler 1 and container 4 of FIG. 1 with the loading member 24 in an inhalation position, an enlarged version providing additional detail is shown in FIG. 10. In the inhalation position the container 4 retained in the holder 26 is located inside the housing 10 having passed through the container opening 64. As the loading member 24 is moved into the inhalation position the opening device 8, in the case a piercing member 70, makes contact with and then breaks through the seal 28 of the container 4 and enters the cavity 90 of the container 4 creating a hole 92 in the seal 28. The seal spreader portion 76 of the piercing member 70 enlarges the hole 92 so that it is bigger than a body region 78 of the piercing member 70. In the inhalation position the piercing member 70 is arranged such that the piercer inlet is located within the container 4 and the hole 92 through the seal 28 is located adjacent to the body region 78 of the piercing member 70 and defines an annular opening into the container 4 around the piercing member 70. In the inhalation position the loading member 24 extends along a portion of the housing 10.

A user can then inhale through the inhaler 1 by placing their mouth around the mouthpiece opening 32 and sucking to create an airflow though the flow channel 6 as shown by arrows 94. As the air passes through the flow directing means 30 the air is directed to flow along the conduit 16 at an angle such that the interaction of the flow with walls defining the conduit 16 cause the air to pass along the conduit in a rotating, or helical, manner (Shown in FIG. 10).

This air flow through the conduit 16 creates a low pressure region at the medicament introduction region 22 due to the restricted portion 17 and so air is drawn through the piercer conduit 72 from the piercer inlet 74. This causes air to be drawn into the cavity 90 of the container 4 through the hole 92 in the seal 28 and into the piercer inlet 74. The air flowing through the cavity 90 entrains the powdered medicament 2 into the airflow and carries said medicament into the conduit 16 where it is carried through the outlet 20 of the conduit 16 and delivered to the user. The powdered medicament 2 may include insulin suitable for treating a patient with diabetes mellitus.

After a user has inhaled through the inhaler 1 the container 4 will contain only residual medicament 2 and needs to be replaced prior to further use. The loading member 24 is pivoted about the loading axis 80 to the loading position as shown in FIG. 6.

FIG. 6 shows a cross section through the inhaler 1 and container 4 of FIG. 1 with the loading member 24 in a loading position after use. The rim 60 of the container 4 extends beyond the fingers 82,84 and allows a user to grip the container 4 for removal. Gaps 83 between the fingers 82, 84, facilitate grasping of the body of the container 4 by exposing an increased surface area of the container 4 for contact. The gaps 83 are preferably aligned with the loading axis 80 as this allows a user to grasp the container 4 more easily as they do not need to insert a finger between the loading member 24 and the housing 10 to reach the gaps 83.

To remove the used (or an unwanted) container from the holder 26 a user must grasp the container 4 and apply a removal force to overcome the force retaining the container 4 in the holder 26. If there was no retaining force then the container 4 may be left in the inhaler 1 when the loading member 24 is moved and since the container 4 is arranged in the container opening 64 it could be difficult to retrieve, or the container 4 may fall from the holder 26 during the handling of the inhaler 1 after initial loading, but prior to moving the loading member 24 into the inhalation position.

Once the container 4 has been removed, the empty loading member 24 can be returned to the inhalation position in which it covers the container opening 64, thereby preventing significant contamination of the inhaler 1 or possible damage to the piercing member 70.

FIG. 7 shows a cut-away view of the front housing 12 showing the slot 38 and cut-outs 50 which allow the snap fit assembly of the inhaler 1.

FIG. 8 shows an enlarged view of the flow directing means 30 showing the angled channels 96 separated by turbulence members 98. The air flowing through the channels 96 is guided to flow through the conduit 16 in a swirling, or helical, manner which can increase turbulence within the air flow and thereby help to entrain and de-agglomerate powdered medicament 2 carried through the flow channel 6.

The flow directing means 30 also helps to prevent foreign objects from entering the flow channel 6 during inhalation which could then be inhaled by a user.

FIG. 9 shows a view from above the loading member 24 and showing the container opening 64. A finger tab 99 which extends from the loading member 24 away from the loading axis 80 is provided to facilitate manual handling of the loading member 24. As shown in previous figures the finger tab 99 is spaced from the housing when the loading member 24 is in the inhalation position and can therefore facilitate the initial moving of the loading member 24 from the inhalation position by allowing a user to more easily grasp the loading member 24, or allowing a finger, or fingernail to be inserted between the finger tab 99 and the housing 10 to initiate the movement.

FIG. 10 shows an enlarged view of FIG. 5 showing the airflow through the inhaler 1 during an inhalation event in more detail.

Arrows 93 show airflow entering the cavity 90 by passing through the hole 92 in the seal 28 formed by the piercing member 70 and arrow 95 indicates the airflow passing through the piercer conduit 72. Arrows 94 generally indicate the flow of air through the conduit 16, and arrows 97 indicate the rotating, or helical, motion created in the air flow by the flow directing means 30. The interaction of the flow directing means 30 and walls defining the conduit 16 cause the airflow to spin substantially about the channel axis 68 to increase turbulence in the air flow.

The inhaler and container may be of any suitable size. The inhaler 1 described above is may be less than about 10 cm long and may be less than about 8 cm long (from mouthpiece opening to intake opening) and is preferably less than about 7 cm long. The container 4 preferably has a maximum diameter across the rim 60 of less than about 2 cm, possibly less than about 1.5 cm and preferably less than about 1.2 cm. The container 4 may extend along the container axis for less than about 2 cm, possibly less than about 1.5 cm and preferably less than about 1.2 cm. The piercing member 70 may extend from the flow channel 6 by less than about 1.5 cm, possible less than about 1 cm, preferably less than about 0.6 cm.

The inhaler 1 may be made from any suitable materials. By way of example, for the inhaler described above the front housing 12, the rear housing 14, the flow directing means 30 and the flow channel 6 may be made of a plastic such as ABS plastic and may include a colorant. The loading member 24 may be made from a polycarbonate plastic and the body 52 of the container 4 may be made from a cyclic olefin polymer.

The loading member 24 and the body 52 of the container 4 may be transparent to allow a user to see into the container 4 more easily to check whether there is significant residual powdered medicament 2, or to see whether there is a container 4 loaded into the inhaler 1.

An example of a suitable powdered medicament 2 is included below, together with a method for manufacturing such a powdered medicament 2.

### Example

| | | Quantity |
|---|---|---|
| Sr. No. | Ingredient | (% w/w) |
| 1 | Recombinant Human Insulin | 60.00% |
| 2 | Mannitol/Trehalose/Raffinose/Sodium | 10.00% |
| | alginate | |
| 3 | Sodium citrate | 27.00% |
| 4 | Glycine | 3.00% |

### Manufacturing Process:

1) Recombinant Human Insulin was dispersed in water
2) Citric acid was added to dissolve the insulin followed by addition of mannitol/trehalose/raffinose/sodium alginate and glycine
3) Sodium citrate was added to the solution obtained in step (2) and pH was adjusted with sodium hydroxide and was spray dried to provide a powered medicament suitable for delivery by the inhaler of the present invention.

It should be understood that the invention has been described above by way of example only and that modifications in detail can be made without departing from the scope of the claims.

1. An inhaler for delivering a powdered medicament to a user from a container, the inhaler comprising:
a flow channel, an opening device and a housing,
the flow channel comprising a conduit extending from an inlet to an outlet and including a medicament introduction region within which the medicament can be introduced into the flow channel,
the inhaler further including a loading member, the loading member including a holder for receiving and retaining the container, the loading member being movable between a loading position, in which the loading member extends away from the housing and the holder is arranged outside of the housing such that the container can be manually loaded into, or removed from, the holder by a user, and an inhalation position in which the container retained in the holder is at least partially within the housing and the powdered medicament stored in the container retained in the holder can be introduced into the flow channel, and
wherein the movement of the loading member from the loading position to the inhalation position causes the opening device to break a seal on the container, thereby exposing the powdered medicament for introduction into the flow channel.

2. An inhaler as claimed in claim 1, in which the opening device comprises a piercing member arranged to pass through and break the seal.

3. An inhaler as claimed in claim 2, in which the piercing member includes a piercer conduit therethrough, the piercer conduit extending from the medicament introduction region of the flow channel to a piercer inlet, the piercer inlet being arranged within the container retained in the holder when the loading member is in the inhalation position allowing the powdered medicament from within said container to be drawn through the piercer conduit and into the flow channel.

4. An inhaler as claimed in claim 3, in which the flow channel extends substantially along a channel axis and in which the piercer conduit extends substantially along a piercer axis, the channel axis being substantially perpendicular to the piercer axis.

5. An inhaler as claimed in claim 1, in which the holder comprises at least two fingers extending from the loading member, said fingers are configured to engage an outer surface of the container arranged in the holder to help retain the container in the holder.

6. An inhaler as claimed in claim 5, in which the fingers are resiliently deformable.

7. An inhaler as claimed in claim 1, in which the container can be retained in the holder by friction forces alone.

8. An inhaler as claimed in claim 1, in which the loading member is coupled to the housing such that it can rotate about an axis between the loading and inhalation positions.

9. An inhaler as claimed in claim 1, in which the housing substantially encloses the flow channel and the housing comprises a mouthpiece opening at or adjacent the outlet, an intake opening at or adjacent the inlet and a container opening for accepting the container retained in the holder when the loading member is moved to the inhalation position.

10. An inhaler as claimed in claim 9, in which the loading member substantially covers the container opening when in the inhalation position.

11. An inhaler as claimed in claim 9, in which the housing comprises a front housing which includes the mouthpiece opening, and a rear housing which includes the intake opening and the container opening.

12. An inhaler as claimed in claim 11, in which the loading member is pivotally coupled to the rear housing.

13. An inhaler as claimed in claim 11, in which the flow channel and the front and rear housings are secured together using snap fit connections.

14. An inhaler as claimed in claim 1, in which the flow channel comprises a restriction in the medicament introduction region.

15. An inhaler as claimed in claim 1, in which the inhaler includes a flow directing means for introducing a rotational flow to air flowing through the flow channel from the inlet to the outlet.

16. An inhaler as claimed in claim 15, in which the flow directing means comprises an insert arranged at, or adjacent to, the inlet end of the flow channel, the insert including a plurality of flow directing channels which extend through the insert, the insert being arranged such that air flowing through the flow channel must pass through the flow directing channels, the flow directing channels being arranged to introduce a rotational flow to said air.

17. An inhaler as claimed in claim 1, in which the medicament introduction region of the conduit is located between the inlet and the outlet.

18. An inhaler as claimed in claim 1, in which the inhaler is a reloadable single unit dry powder inhaler.

19. An inhaler kit comprising the inhaler and the container as claimed in claim 1, and
the container comprising a body and a seal,
wherein the body includes a cavity containing the powdered medicament, and the seal closes the cavity.

20. An inhaler kit as claimed in claim 19, in which the powdered medicament comprises insulin.

21. An inhaler kit as claimed in claim 20, in which the insulin comprises human insulin.

22. An inhaler kit as claimed in claim 19, in which the powdered medicament is spray dried and comprises sodium citrate.

23. A method for using the inhaler kit as claimed in claim 19, the method including the steps of:
manually moving the loading member into a loading position;
loading the container into the holder and using the holder to retain the container;
moving the loading member into the inhalation position in which the powdered medicament stored within the container can be introduced into the flow channel;
causing the opening device to break the seal on the container as the loading member is moved from the loading position to the inhalation position;
causing air flow through the flow channel from the inlet to the outlet;
allowing powdered medicament from the container to enter the flow channel and become entrained in the air flow as a result of the air flow through the flow channel;
manually moving the loading member from the inhalation position to the loading position while retaining the container in the holder; and
manually removing the container from the holder.

24. A method for loading the container into the inhaler as claimed in claim 1, the method comprising:
providing the container comprising a body and a seal, wherein the body includes a cavity containing the powdered medicament and the seal closes the cavity;
manually moving the loading member into the loading position;
loading the container into the holder and using the holder to retain the container;
moving the loading member into the inhalation position in which the powdered medicament stored within the container can be introduced into the flow channel; and
causing the opening device to break the seal on the container as the loading member is moved from the loading position to the inhalation position.

25. A method for removing the opened container from the inhaler as claimed in claim 1, the inhaler having the opened container retained in the holder, the container comprising a body and a broken seal, wherein the body includes a cavity and the seal closed the cavity before it was broken, and the method comprising the steps of:
manually moving the loading member from the inhalation position to the loading position while retaining the container in the holder; and
manually removing the container from the holder.

26. A method for treating a patient diagnosed with diabetes mellitus, the method comprising:
administering to the patient a powdered medicament comprising insulin from an inhaler, the inhaler comprising a flow channel, an opening device and a housing, the flow channel comprising a conduit extending from an inlet to an outlet and including a medicament introduction region within which the medicament can be introduced into the flow channel, the inhaler including a loading member, the loading member including a holder for receiving and retaining a medicament container, the loading member being manually movable between a loading position, in which the loading member extends away from the housing and the holder is arranged outside of the housing such that container can be manually loaded into, or removed from, the holder, and an inhalation position in which the container retained in the holder is at least partially within the housing and the powdered medicament stored in the container retained in the holder can be introduced into the flow channel, wherein the movement of the loading member from the loading position to the inhalation position causes the opening device to break a seal on the container, thereby exposing the powdered medicament for introduction into the flow channel.

27. A method, comprising:
delivering by an inhaler a powdered medicament comprising insulin for the treatment of at least one patient diagnosed with diabetes mellitus for the control of hyperglycemia, the inhaler comprising a flow channel, an opening device and a housing, the flow channel comprising a conduit extending from an inlet to an outlet and including a medicament introduction region within which the medicament can be introduced into the flow channel, the inhaler including a loading member, the loading member including a holder for receiving and retaining a medicament container, the loading member being manually movable between a loading position, in which the loading member extends away from the housing and the holder is arranged outside of the housing such that the container can be manually loaded into, or removed from, the holder, and an inhalation position in which container retained in the holder is at least partially within the housing and the powdered medicament stored in the container retained in the holder can be introduced into the flow channel, wherein the movement of the loading member from the loading position to the inhalation position causes the opening device to break a seal on the container, thereby exposing the powdered medicament for introduction into the flow channel.

28. (canceled)
